(19) United States
(12) Patent Application Publication
(10) Pub. No.: US 2019/0221294 A1
(43) Pub. Date: Jul. 18, 2019

(54) TIME SERIES DATA PROCESSING DEVICE, HEALTH PREDICTION SYSTEM INCLUDING THE SAME, AND METHOD FOR OPERATING THE TIME SERIES DATA PROCESSING DEVICE

(71) Applicant: ELECTRONICS AND TELECOMMUNICATIONS RESEARCH INSTITUTE, Daejeon (KR)

(72) Inventors: Ho-Youl JUNG, Daejeon (KR); Hwin Dol PARK, Daejeon (KR); Myung-Eun LIM, Daejeon (KR); Jae Hun CHOI, Daejeon (KR); Youngwoong HAN, Daejeon (KR)

(73) Assignee: ELECTRONICS AND TELECOMMUNICATIONS RESEARCH INSTITUTE, Daejeon (KR)

(21) Appl. No.: 16/213,740

(22) Filed: Dec. 7, 2018

(30) Foreign Application Priority Data
Jan. 12, 2018 (KR) 10-2018-0004702
Oct. 2, 2018 (KR) 10-2018-0117899

(51) Int. Cl. G16H 10/60; G16H 50/20

(52) U.S. Cl. CPC G16H 10/60; G16H 50/20

(57) ABSTRACT

The inventive concept relates to a multi-dimensional time series data processing device, a health prediction system including the same, and a method of operating the time series data processing device. A time series data processing device according to an embodiment of the inventive concept includes a network interface, a data generator, a predictor, and a processor. The network interface receives the first time series data having the first type. The data generator generates second time series data having a second type based on the first time series data. The predictor generates prediction data based on the first time series data and the second time series data.

### CROSS-REFERENCE TO RELATED APPLICATIONS

This U.S. non-provisional patent application claims priority under 35 U.S.C. § 119 of Korean Patent Application Nos. 10-2018-0004702, filed on Jan. 12, 2018, and 10-2018-0117899, filed on Oct. 2, 2018, the entire contents of which are hereby incorporated by reference.

### BACKGROUND

The present disclosure herein relates to the processing of time series data and the construction of a generation model therefor, and more particularly, to a time series data processing device, a health prediction system including the same, and a method for operating the time series data processing device.

The development of various technologies including medical technology improves human standard of living and increases human life span. However, changes in lifestyle and erroneous eating habits due to technological development are causing various diseases. In order to lead a healthy life, there is a need to anticipate the future health conditions from treating the current disease. Future health conditions may be predicted by analyzing the trend of time series medical data over time.

The development of industrial technology and information and communication technologies is creating a significant amount of information and data. In recent years, technologies such as artificial intelligence that provides various services by learning an electronic device such as a computer using such a large amount of information and data are emerging. In particular, in order to predict future health conditions, methods are suggested to construct models for processing or analyzing various time series medical data. For example, time series medical data may be provided in different types (or modality) depending on collected devices or institutions. To improve the prediction accuracy of future health conditions, there is a need for effectively utilizing models constructed to effectively process different types of time series medical data or to use different types of time series medical data.

### SUMMARY

The present disclosure is to provide a time series data processing device for predicting future time data using time series data having different types or modalities, a health prediction system including the same, and a method for operating the time series data processing device.

An embodiment of the inventive concept provides a time series data processing device including: a network interface configured to receive first time series data corresponding to a previous time of a target time point, the first time series data having a first type; a data generator configured to generate a second time series data corresponding to a previous time of the target time point based on the first time series data, the second time series data having a second type; a predictor configured to generate prediction data corresponding to a later time of the target time point based on the first time series data and the second time series data; and a processor configured to control the data generator and the predictor.

In an embodiment, the first time series data may be a grouped electronic medical record generated at a plurality of time points preceding the target time point, wherein the data generator may generate the second time series data corresponding to a virtual personal health record based on the electronic medical record.

In an embodiment, the data generator may generate the second time series data based on a generation model learned by third time series data having the first type and fourth time series data having the second type, wherein the network interface may receive the third and fourth time series data before receiving the first time series data.

In an embodiment, the data generator may include: a generator configured to generate fifth time series data having the second type based on the third and fourth time series data; and a discriminator configured to determine whether the fifth time series data is data generated from the generator.

In an embodiment, until the discriminator does not determine the fifth time series data as data generated from the generator, a weight of the generation model may be adjusted.

In an embodiment, the data generator may include: an embedder configured to convert each of the third time series data and the fourth time series data to have the same type, wherein the generation model may be learned based on the converted third and fourth time series data.

In an embodiment, the embedder may convert the first time series data to have the same type as the converted third and fourth time series data, wherein the generation model may generate the second time series data based on the converted first time series data.

In an embodiment, the first time series data may include first feature data that is numerical data and second feature data that is non-numerical data, wherein the data generator may convert the second feature data into numerical data and generate the second time series data based on the first feature data and the second feature data converted into the numerical data.

In an embodiment, the second time series data may be time series data having a predetermined reference time interval.

In an embodiment of the inventive concept, a health prediction system includes: a collection device configured to collect first time series data corresponding to an electronic medical record; and a medical data processing device configured to generate second time series data corresponding to a virtual personal health record and having a reference time interval based on the first time series data, and generate prediction data of a future time point based on the first time series data and the second time series data.

In an embodiment, the medical data processing device may include: a personal health record generator configured to generate the second time series data based on the first time series data; and a health predictor configured to generate the electronic medical record of the future time point based on the first and second time series data.

In an embodiment, the health predictor may generate the prediction data corresponding to the electronic medical record of the future time point, based on a prediction model for analyzing a change trend of the first time series data with respect to time and a change trend of the second time series data with respect to time in parallel.

In an embodiment, the system may further include a second collection device configured to collect third time series data corresponding to the second electronic medical record and a fourth time series data corresponding to a personal health record measured from a personal health sensor, wherein the medical data processing device may learn a generation model based on the third and fourth time series data and input the first time series data to the generation model to generate the second time series data.

In an embodiment, the medical data processing device may input the third and fourth time series data to the generation model to generate fifth time series data corresponding to a virtual personal health record, and learn the generation model until it is not determined whether the fifth time series data is the virtual personal health record or the measured personal health record.

In an embodiment, the medical data processing device may convert each of the third time series data and the fourth time series data to have the same type and inputs them to the generation model.

In an embodiment of the inventive concept, provided is a method of operating a time series data processing device performed by a processor. The method includes: receiving first time series data generated to have a first type at past time points, through a network interface; embedding the first time series data to generate input data; inputting the input data to a generation model to generate second time series data corresponding to past time points having a reference time interval and having a second type; and generating prediction data of a future time point based on the first time series data and the second time series data.

In an embodiment, the method may further include, before receiving the first time series data, learning the generation model, based on third time series data collected to have the first type and fourth time series data collected to have the second type.

In an embodiment, the learning of the generation model may include: receiving the third and fourth time series data through the network interface; generating learning data by embedding the third and fourth time series data to have the same type; inputting the learning data to the generation model to generate fifth time series data corresponding to past time points having the reference time interval and having the second type; and determining whether the fifth time series data is time series data received through the network interface or time series data generated from the generation model.

In an embodiment, the learning of the generation model may further include, when the fifth time series data is determined as time series data generated from the generation model, adjusting a weight of the generation model.

In an embodiment, the generating of the prediction data may include: generating first intermediate data based on a change trend of the first time series data with respect to time; generating second intermediate data based on a change trend of the second time series data with respect to time; and calculating the prediction data based on the first intermediate data and the second intermediate data.

### BRIEF DESCRIPTION OF THE FIGURES

The accompanying drawings are included to provide a further understanding of the inventive concept, and are incorporated in and constitute a part of this specification. The drawings illustrate exemplary embodiments of the inventive concept and, together with the description, serve to explain principles of the inventive concept. In the drawings:

FIG. 1 is a view showing a health prediction system according to an embodiment of the inventive concept;

FIG. 2 is a view showing a health prediction system according to an embodiment of the inventive concept;

FIG. 3 is a block diagram for specifically explaining the operation of the PHR generator of FIG. 2 in the learning operation;

FIG. 4 is a block diagram for specifically explaining the operation of the PHR generator of FIG. 2 in the generation operation;

FIG. 5 is a view for explaining the embedder of FIG. 3 and FIG. 4 in detail;

FIG. 6 is an exemplary block diagram of the medical data processing device of FIG. 2;

FIG. 7 is a view for explaining a process of learning a generation model by the medical data processing device of FIGS. 2 and 6; and

FIG. 8 is a view for explaining a process in which the medical data processing device of FIGS. 2 and 6 operates based on a learned generation model.

### DETAILED DESCRIPTION

In the following, embodiments of the inventive concept will be described in detail so that those skilled in the art easily carry out the inventive concept.

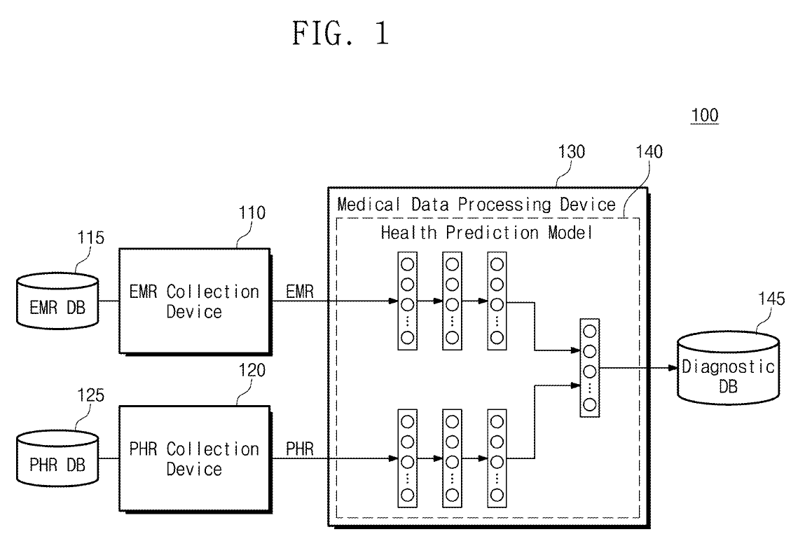

FIG. 1 is a view showing a health prediction system according to an embodiment of the inventive concept. Referring to FIG. 1, a health prediction system 100 includes an electronic medical record collection device 110 (hereinafter referred to as an EMR collection device), an EMR database 115, a personal health record collection device 120 (hereinafter referred to as a PHR collection device), a PHR database 125, a medical data processing device 130, and a diagnostic database 145.

The EMR collection device 110 may collect an electronic medical record (EMR) indicating user's health conditions generated by diagnosis, treatment, or medication prescription at a medical institution. EMR is generated when visiting a medical institution and may include feature data generated based on diagnostic, therapeutic, or medication-prescribed features (e.g., blood pressure, cholesterol levels, and the like). For example, the feature data may be data measured by a test such as blood pressure or data representing the degree of a disease such as atherosclerosis.

The EMR collection device 110 may collect EMRs from a medical institution, such as a public institution or hospital, or from an EMR database 115, which is constructed by a management company or institution designated by a corresponding medical institution. The EMR is generated each time a user visits a medical institution, and may be grouped and managed in a time series for each user in the EMR database 115. The EMR database 115 may be implemented in a server or storage medium.

The PHR collection device 120 may collect a personal health record (PHR) managed and generated by an individual such as a user. The PHR may be generated from medical data measured from individual health sensors that are individually provided, such as a home body scanner, and may include feature data generated based on features measured by the personal health sensor. Here, the defined PHR will be understood as time series medical data measured directly by the user using a personal health sensor, not a medical institution such as a hospital.

The PHR collection device 120 may collect PHRs from the PHR database 125 established by a user or a management company or institution designated by the user. The PHR may be generated each time a user uses a personal health sensor and may be grouped and managed in a time series in the PHR database 125. The PHR database 125 may be implemented in a server or storage medium.

Because EMR is generated by specialized medical institutions using precise medical equipment, it may be highly accurate in diagnosing, evaluating, and predicting personal health conditions compared to PHR. However, the EMR is generated as the user visits the medical institution directly. Thus, it may be difficult to obtain sufficient medical data in consideration of the cost of visiting a medical institution, the physical distance, and the constantly changing purpose of the visit. In addition, since EMR is generated by irregular visits, it may be difficult to obtain regular medical data in time series.

Since the PHR is generated by using a personal health sensor which is easy to access by the user, it may be generated regularly in time series compared to the EMR. In addition, since it is convenient to continuously inspect the same feature, the feature data included in the PHR may be less missed than the EMR over time. However, since PHR is not obtained with precision equipment as compared to EMR, it has low accuracy in diagnosing, evaluating, and predicting personal health condition. In addition, since the PHR database 125 is not universally established at present and the data measured by the personal health sensor or the like is not managed by the medical institution in a database, the absolute amount of time series medical data corresponding to the PHR is insufficient compared to the EMR.

The medical data processing device 130 may analyze both the above-described EMR and PHR to predict a user's health condition at a future time. In this case, the medical data processing device 130 may generate the prediction data considering both the accuracy of the EMR and the time series regularity of the PHR. Here, the prediction data may be the predicted value of the EMR of the specified future time point, but is not limited thereto, and may be PHR or other types of medical data. The medical data processing device 130 may receive the EMR from the EMR collection device 110 and receive the PHR from the PHR collection device 120.

The medical data processing device 130 may construct a health prediction model 140 for predicting future health conditions using EMR and PHR having different types or modalities. The health prediction model 140 may be generated by learning various EMRs and PHRs. The health prediction model 140 may be layered into a plurality of layers. For example, the health prediction model 140 may be a neural network model, but not limited thereto, and various learning models capable of performing machine learning may be applied to the health prediction model 140.

The health prediction model 140 receives the EMR and the PHR in parallel, and analyzes the EMR and the PHR, respectively. For example, the health prediction model 140 may generate the first intermediate data based on the change trend of the EMR over time, and may generate the second intermediate data based on the change trend of the PHR over time. The health prediction model 140 may finally generate the prediction data by merging the first intermediate data and the second intermediate data to analyze the relationship and pattern between similar features. That is, the health prediction model 140 may include a layer for shared representations of the two modalities.

The prediction data generated by the health prediction model 140 may be constructed in a diagnostic database 145. The prediction data may be grouped and managed for each user in the diagnostic database 145. Illustratively, to predict the user's health condition at any future time, the diagnostic database 145 may manage the trend information of the future health condition according to the analyzed time based on the health prediction model 140 and may further manage the EMR and PHR, that is, raw data. The diagnostic database 145 may be implemented in a server or storage medium.

By implementing the health prediction model 140 to use both EMR and PHR, the prediction accuracy of the future health condition may be improved. However, when the medical data processing device 130 in which the health prediction model 140 is constructed is used, the amount of data of any one of different types of time series data may be insufficient. In particular, even if the user regularly uses the personal health sensor in the time series, since the PHR is often not databaseized like the EMR, it is difficult to obtain enough time series data corresponding to the past time points. Also, since PHR is generated from an individual, the cost for collecting PHR is increased, and data collection constraints are followed. In addition, unique ethical issues, legal issues, and personal privacy issues in the medical field make it difficult to collect medical data. The following description shows a system and method for solving the problem in the already constructed multi-modality-based health prediction model 140 based on retrospective research.

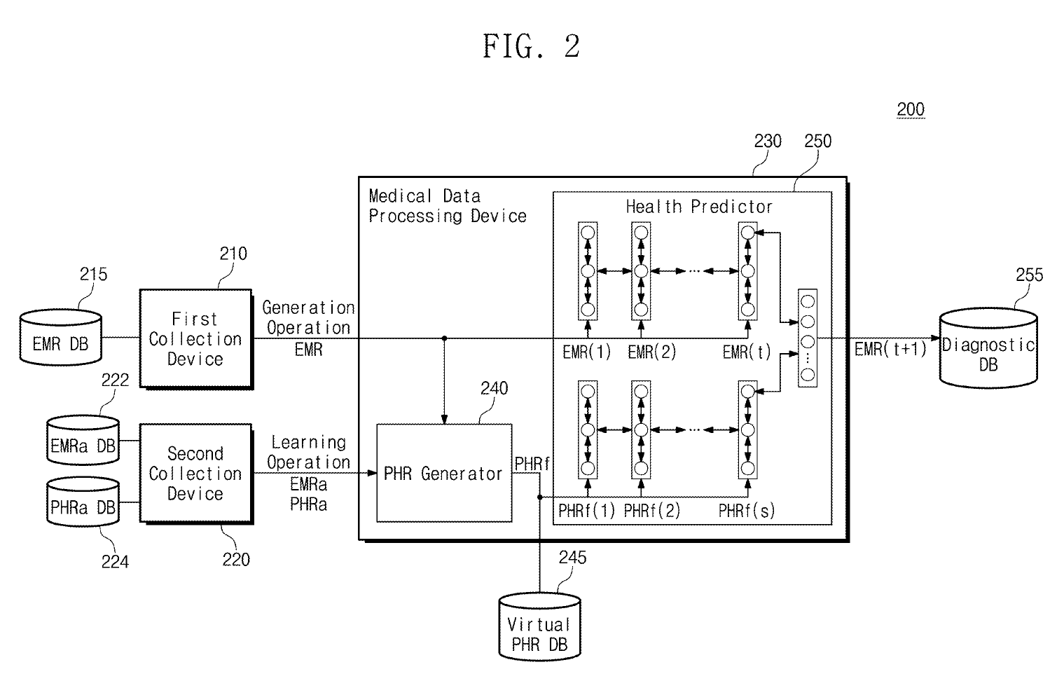

FIG. 2 is a view showing a health prediction system according to an embodiment of the inventive concept. Referring to FIG. 2, a health prediction system 200 includes a first collection device 210, an EMR database 215, a second collection device 220, a learning EMR database 222, a learning PHR database 224, a medical data processing device 230, a virtual PHR database 245, and a diagnostic database 255. The health prediction system 200 of FIG. 2 will be understood as an exemplary configuration for generating a virtual PHR to predict future health conditions, and the structure of the health prediction system 200 will not be limited thereto.

The first collection device 210 may collect EMRs, which are time series data, to predict the future health condition of the user. The first collection device 210 may collect the EMR from the EMR database 215. The EMR database 215 may correspond to the EMR database 115 of FIG. 1. As described above, by using different types of EMR and PHR, the prediction accuracy of future health condition may be improved. However, the amount of data is insufficient because the PHR of the past time is often not databaseized, and there are cost, legal, and procedural difficulties in collecting PHRs to utilize health prediction models. For convenience of explanation, it is assumed that the PHR for predicting a future health condition is not collected in the health prediction system 200 of FIG. 2. The EMR is used to generate the virtual PHR.

The second collection device 220 may collect learning EMR EMRa and learning PHR PHRa, which are time series data, in order to learn a generation model for generating a virtual PHR. The second collection device 220 may collect the learning EMR EMRa from the learning EMR database 222 and collect the learning PHR PHRa from the learning PHR database 224. The learning EMR EMRa and the learning PHR PHRa may have different types and may be generated from different institutions or medical devices, but may be integrally managed. For example, a hospital managing the learning EMR EMRa may receive and manage the learning PHR PHRa generated from a user's personal health sensor. The EMR database 215 may be managed by a medical institution other than the institution managing the learning EMR database 222 and the learning PHR database 224, but is not limited thereto. Before the first collection device 210 provides the EMR to the medical data processing device 230, the second collection device 220 provides a learning EMR EMRa and a learning PHR PHRa to the medical data processing device 230.

The medical data processing device 230 is a time series data processing device for analyzing EMR and PHR to predict a user's health condition at a future time. However, as shown in FIG. 2, when there is no PHR for predicting a future health condition, or when the PHR is insufficient, the medical data processing device 230 may generate a virtual PHR PHRf. The medical data processing device 230 may include a PHR generator 240 and a health predictor 250.

The PHR generator 240 is a data generator for generating a virtual PHR PHRf which is time series data. For this, the PHR generator 240 may construct a generation model. In the learning operation, the generation model may be generated by learning the learning EMR EMRa and the learning PHR PHRa. For example, the generation model may be implemented as a Generative Adversarial Network (GAN), but not limited thereto, and various models capable of performing machine learning may be applied to the generation model. The specific learning operations of the PHR generator 240 are described below.

In the generation operation, the PHR generator 240 generates a virtual PHR PHRf based on the EMR. The EMR is inputted into the learned generation model. The generation model generates a virtual PHR PHRf having a different type from the EMR. An EMR has a stereotyped type represented by a numerical value, a non-numeric value such as a sign or a symbol, depending on the feature, and the PHR may have a type that, unlike the EMR, is represented by a numerical value measured by a personal health sensor. Generation models may generate time series data with different types of EMR based on learning results. In addition, the generation model may generate a virtual PHR PHRf having a regular time interval, unlike the temporally irregular EMR. The virtual PHR PHRf may be time series data having a reference time interval. For example, the reference time interval may be a predetermined time interval considering the prediction accuracy and the processing speed of the health predictor 250 for the future health condition. The virtual PHR PHRf may be constructed and managed in the virtual PHR database 245. The specific generation operations of the PHR generator 240 are described below.

The health predictor 250 is a predictor for predicting future health conditions using different types of EMR and virtual PHR PHRf. For this, the health predictor 250 may construct a prediction model. The prediction model may be generated by learning various EMRs and PHRs, like the health prediction model 140 of FIG. 1. The prediction model may be implemented as a circular neural network, such as a recurrent neural network (RNN) or a long-short term memory (LSTM), as shown in FIG. 2. The prediction model may process time series data such as EMR or virtual PHR PHRf sequentially according to time, but may process the time series data such that the EMR or virtual PHR PHRf corresponding to the previous time point is reflected in the EMR or virtual PHR PHRf corresponding to the next time point.

The health predictor 250 receives the EMR and the virtual PHR PHRf in parallel, and analyzes the EMR and the virtual PHR PHRf, respectively. Illustratively, the EMR may be time series data corresponding to irregular t time points, and the virtual PHR PHRf may be time series data corresponding to s regular past time points having a reference time interval. The health predictor 250 may generate the first intermediate data based on the change trend of the EMR over time, and may generate the second intermediate data based on the change trend of the virtual PHR PHRf over time. The health predictor may generate the prediction data based on the first intermediate data and the second intermediate data, and for this, the prediction model may include layers for shared representations of the two modalities. Illustratively, although it is shown that the prediction data is an EMR corresponding to a future t+1 time point, it is not limited thereto and may have various types that may represent future health conditions. The prediction data may be constructed and managed in the diagnostic database 255.

That is, the health prediction system 200 does not propose a prospective research-based solution, such as measuring additional PHR, in a multi-modality based prediction model that is already established. As a retrospective research-based solution, the health prediction system 200 generates a virtual PHR PHRf instead of collecting the PHR. Thus, cost, legal and procedural difficulties due to the additional collection of PHRs may be solved.

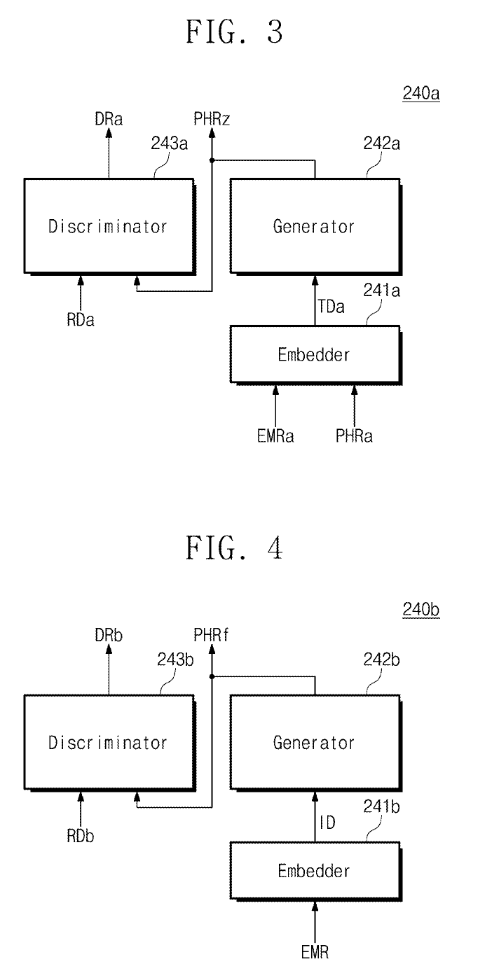

FIG. 3 is a block diagram for specifically explaining the operation of the PHR generator of FIG. 2 in the learning operation. Referring to FIG. 3, the PHR generator 240a includes an embedder 241a, a generator 242a, and a discriminator 243a. The PHR generator 240a corresponds to the PHR generator 240 of FIG. 2. The PHR generator 240a is described as being implemented based on a generative adversarial network (GAN). For convenience of explanation, referring to the reference numerals of FIG. 2, FIG. 3 will be described.

The embedder 241a may convert each of the learning EMR EMRa and the learning PHR PHRa inputted from the second collection device 220 to have the same type. The learning EMR EMRa, which is the time series data of the electronic medical record, and the learning PHR PHRa, which is the time series data of the personal health record, are generated in different types. For example, the learning EMR EMRa may be mixed with numerical data and non-numerical data, and the learning PHR PHRa may include only numerical data. In addition, the learning EMR EMRa and the learning PHR PHRa may have different dimensions and may express features in different ways. The embedder 241a may embed the learning EMR EMRa and the learning PHR PHRa, respectively, and convert them into the same vector form. For example, the embedder 241a may quantify the learning EMR EMRa and the learning PHR PHRa using the Word2Vec method. However, the inventive concept is not limited thereto, and the learning EMR EMRa and the learning PHR PHRa may be converted to an EMR type, a PHR type, or a different type from EMR or PHR.

The embedder 241a may convert the learning EMR EMRa and the learning PHR PHRa to generate learning data TDa which is time series data. The embedder 241a converts the learning EMR EMRa and the learning PHR PHRa to have the same type and outputs them as time series data arranged over time. The learning data TDa is inputted to the generator 242a.

The generator 242a may generate virtual time series data PHRz based on the learning data TDa. The virtual time series data PHRz may have the same type as the PHR. However, the inventive concept is not limited thereto. For example, the virtual time series data PHRz may have the same type as the vector type converted by the embedder 241a. The generator 242a may generate time series data corresponding to virtual past time points but virtual past time points may be set to have a reference time interval. The virtual time series data PHRz is inputted to the discriminator 243a.

The generator 242a may be a neural network model constructed through learning, but not limited thereto, and various learning models capable of performing machine learning may be applied to the generator 242a. For example, in order to process learning data TDa which is time series data, the generator 242a may be implemented as a circular neural network such as a Recurrent Neural Network (RNN) or a Long-Short Term Memory (LSTM). In the learning operation, the weight of the generator 242a may be adjusted. Since the generator 242a generates the virtual time series data PHRz using the learning data TDa considering the learning EMR EMRa, it generates time series data with high relevance to EMR.

The discriminator 243a may determine whether the virtual time series data PHRz is virtual data generated from the generator 242a. The discriminator 243a may receive virtual time series data PHRz and real data RDa. The discriminator 243a may perform an operation of distinguishing virtual time series data PHRz from real data RDa. For example, if the virtual time series data PHRz has the same type as the PHR, the real data RDa may include a learning PHR PHRa, or may include a learning EMR EMRa converted into a PHR type and a learning PHR PHRa, by the embedder 241a or a separate configuration. For example, if the virtual time series data PHRz has the same type as the vector type converted by the embedder 241a, the real data RDa may include the learning data TDa. As an example, the real data RDa may include PHRs collected in a previous learning operation.

The discriminator 243a may generate the discrimination result data DRa based on the result of discriminating that the virtual time series data PHRz is virtual data. The discriminator 243a may generate the determination result data DRa based on the normal distribution of the real data RDa and the normal distribution of the virtual time series data PHRz. For example, the discrimination result data DRa may have a value between 0 and 1, which is generated according to a result of discrimination of virtual data based on a sigmoid function or the like. At this time, when the normal distribution of the real data RDa and the normal distribution of the virtual time series data PHRz coincide with each other, the determination result data DRa having a value of 0.5 may be outputted.

Based on a result of discrimination, when the real data RDa and the virtual time series data PHRz are distinguished, the weight of the generator 242a may be adjusted. Further, the operation of generating the virtual time series data PHRz may be repeated again. Until the discriminator 243a may not distinguish the real data RDa from the virtual time series data PHRz, the generator 242a may repeat the operation of adjusting the weight and generating virtual time series data PHRz. As a result, the generator 242a may be learned to generate virtual time series data PHRz having a normal distribution like the real data RDa. The discriminator 243a may be a neural network model constructed through learning, but not limited thereto, and various learning models capable of performing machine learning may be applied to the discriminator 243a.

FIG. 4 is a block diagram for specifically explaining the operation of the PHR generator of FIG. 2 in the generation operation. Referring to FIG. 4, the PHR generator 240b includes an embedder 241b, a generator 242b, and a discriminator 243b. The PHR generator 240b corresponds to the PHR generator 240 of FIG. 2. The PHR generator 240b is described as being implemented on a GAN basis. For convenience of explanation, referring to the reference numerals of FIG. 2, FIG. 4 will be described.

The embedder 241b may convert the EMR inputted from the first collection device 210. Since the embedder 241b is substantially the same as the embedder 241a of FIG. 3, it may convert the EMR to a type identical to the type in which the learning EMR EMRa and the learning PHR PHRa are converted. The embedder 241b may embed the EMR and convert it into a vector form. Illustratively, although it is assumed that no separate PHR is inputted in the generation operation, a PHR having a data amount less than the amount of data included in the EMR may be inputted to the embedder 241b together. In this case, EMR and PHR may be converted to the same type. Based on embedding results, input data ID is generated.

The generator 242b may generate the virtual PHR PHRf based on the input data ID. The generator 242b that learns in the learning operation may generate a virtual PHR PHRf like the PHR provided from the collection device. The virtual PHR PHRf may be time series data having a reference time interval. Since the generator 242b generates the virtual PHR PHRf using the input data ID generated by the EMR, it may generate a virtual PHR PHRf highly related to the EMR.

The discriminator 243b may determine whether the virtual PHR PHRf is virtual data generated from the generator 242b. That is, the PHR generator 240b may continuously perform the learning operation even in the generation operation. For this, the discriminator 243b may perform an operation of distinguishing the virtual PHR PHRf from the real data RDb. For example, the real data RDb may include the real data RDa provided in the learning operation of FIG. 3. The discriminator 243b may generate the discrimination result data DRb based on the discrimination result. Based on a result of discrimination, when the real data RDb and the virtual PHR PHRf are distinguished, the weights of the generator 242b may be adjusted again and the virtual PHR PHRf may be regenerated based on the adjusted weight. If the real data RDb and the virtual PHR (PHRf) are not distinguishable, the virtual PHR PHRf may be outputted to the health predictor 250.

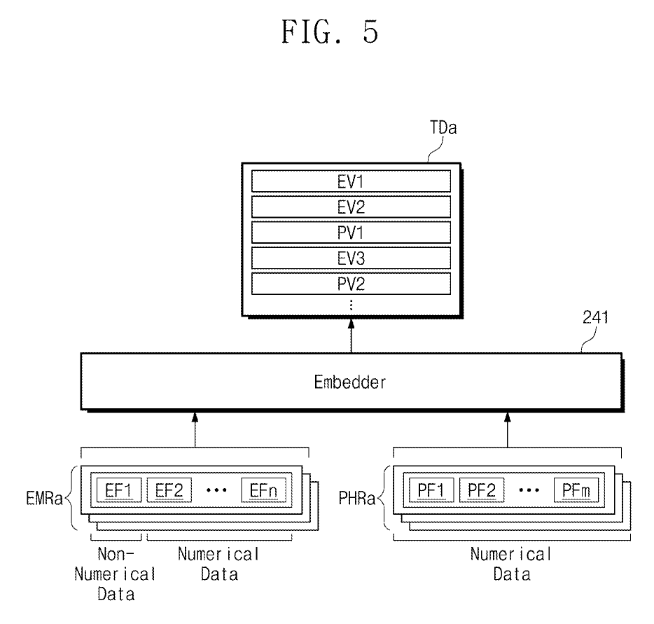

FIG. 5 is a view for explaining the embedder of FIGS. 3 and 4 in detail. Referring to FIG. 5, the embedder 241 converts the learning EMR EMRa and the learning PHR PHRa to have the same type. Each of the learning EMR EMRa and the learning PHR PHRa may be time series data collected from the second collection device 220 of FIG. 2. Each of the learning EMR EMRa and the learning PHR PHRa may be time series data having different types. The learning EMR EMRa may include a plurality of EMRs generated at a plurality of past time points according to a visit of a medical institution. The learning PHR PHRa may include a plurality of PHRs generated according to the use of a personal health sensor at a plurality of past time points.

Each of the plurality of EMRs may include first to n-th EMR feature data EF1 to EFn. The first to n-th EMR feature data EF1 to EFn are generated by individual diagnoses, treatments, or medication prescriptions received at a medical institution. Each of the plurality of EMRs may include numerical data and non-numerical data. Illustratively, it is assumed that the first EMR feature data EF1 is non-numerical data and the second to n-th EMR feature data EF2 to EFn are numerical data. For example, feature data, such as disease code data generated based on disease diagnosis, or medication code data generated based on a drug prescription, may be non-numerical data in code form, such as E02.31. For example, the feature data generated on the basis of the inspection result of the body composition may be numerical data such as a blood sugar value, feature data including information of a category type (−, +, ++, etc.) such as hematuria characteristic may be non-numerical data.

Each of the plurality of PHRs may include first to m-th PHR feature data PF1 to PFm. The first to m-th PHR feature data PF1 to PFm are generated by biometric information measured by the user's personal health sensor. Each of the first to m-th PHR feature data PF1 to PFm may be numerical data. For example, the feature data generated based on the measurement results of the body composition, etc. may be numerical data such as blood sugar values.

The embedder 241 may convert each of the learning EMR EMRa and the learning PHR PHRa into a vector format having the same type. The embedder 241 may embed non-numerical data and numerical data in the learning EMR (EMRa) and quantify them. The embedder 241 may convert the digitized learning EMR EMRa into a vector type such as the first to third EMR vector data EV1 to EV3. Each of the first to third EMR vector data EV1 to EV3 corresponds to the EMRs generated at a specific time point in the past. Although not shown in detail, each of the first to third EMR vector data EV1 to EV3 may represent features corresponding to the first to n-th EMR feature data EF1 to EFn as a vector type.

The embedder 241 may embed the learning PHR PHRa and convert it into a vector type such as the first to second PHR vector data PV1 to PV2. Each of the first and second PHR vector data PV1 to PV2 corresponds to PHRs generated at a specific time point in the past. Although not shown in detail, each of the first and second PHR vector data PV1 to PV2 may represent features corresponding to the first to m-th PHR feature data PF1 to PFm as a vector type. As the similarity between features is greater, data having a vector type may be generated to be located closer to a vector space.

The embedder 241 may generate learning data TDa, which is time series data, as a result of embedding the learning EMR EMRa and the learning PHR PHRa, respectively. The learning data TDa may include first to third EMR vector data EV1 to EV3 and first to second PHR vector data PV1 to PV2. The embedder 241 may align the training data TDa in the order of time and output it to the generators 242a and 242b. For example, the EMR corresponding to the first EMR vector data EV1 may be generated earlier, and the EMR corresponding to the second EMR vector data EV2, the PHR corresponding to the first PHR vector data PV1, and the like may be sequentially generated.

Since the embedder 241 converts time series data having different types to have the same type, the PHR generator 240 may generate virtual time series data in consideration of various types. Also, the embedder 241 outputs the learning data TDa (or the input data ID in FIG. 4) in the order of time sequence, the PHR generator 240 may easily analyze the change of the learning data TDa (or the input data ID in FIG. 4) over time.

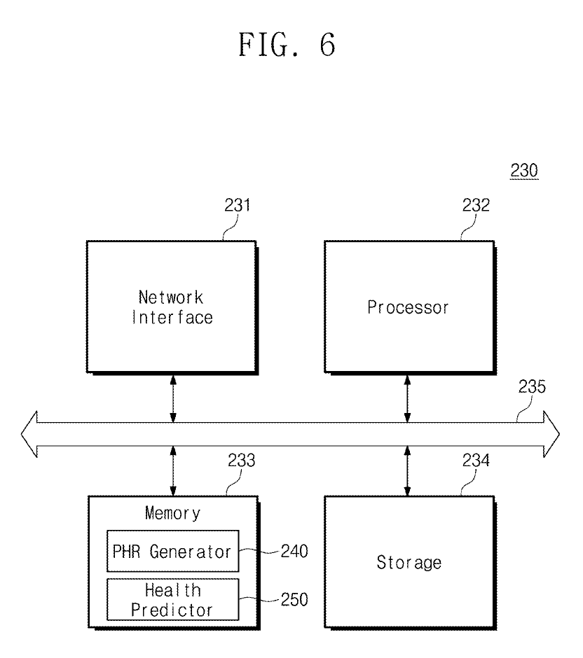

FIG. 6 is an exemplary block diagram of the medical data processing device of FIG. 2. The block diagram of FIG. 6 will be understood as an exemplary configuration for generating a virtual PHR and for predicting future health conditions based on the collected EMR and virtual PHR. Accordingly, the configuration of the medical data processing device 230 will not be limited thereto. Referring to FIG. 6, the medical data processing device 230 may include a network interface 231, a processor 232, a memory 233, a storage 234, and a bus 235. Illustratively, the medical data processing device 230 may be implemented as a server, but is not limited thereto.

The network interface 231 is configured to receive time series medical data of the EMR or PHR type provided from the first collection device 210 or the second collection device 220 of FIG. 2. The network interface 231 may provide the received time series medical data to the processor 232, the memory 233 or the storage 234 through the bus 235. In addition, the network interface 231 may be configured to provide prediction results of future health conditions generated in response to the received time series medical data to a terminal (not shown) through a network.

The processor 232 may function as a central processing unit of the medical data processing device 230. The processor 232 may perform the control and computation operations required to implement virtual time series data generation of the medical data processing device 230 and prediction of future health conditions. For example, according to the control of the processor 232, the network interface 231 may receive time series medical data from the outside. Under the control of the processor 232, a computation operation may be performed to generate a generation model for generating a virtual PHR or a prediction model for predicting a future health condition. Under the control of the processor 232, virtual PHR or prediction data may be calculated. The processor 232 may operate utilizing the computation space of the memory 233 and may read files and executable files of the application for running the operating system from the storage 234. The processor 232 may execute the operating system and various applications.

The memory 233 may store data and process codes processed or to be processed by the processor 232. For example, the memory 233 may store time series medical data provided from the network interface 231, information for performing an operation of generating a virtual PHR, information for calculating prediction data, or information for constructing a generation model or a prediction model and the like. The memory 233 may be used as a main memory of the medical data processing device 230. The memory 233 may include a dynamic random access memory (DRAM), a static random access memory (SRAM), a phase change RAM (PRAM), a magnetic RAM (MRAM), a ferroelectric RAM (FeRAM), and so on.

The memory 233 may include a PHR generator 240 and a health predictor 250. The PHR generator 240 and the health predictor 250 may be part of the computing space of memory 233. In this case, the PHR generator 240 and the health predictor 250 may be implemented in firmware or software. For example, the firmware may be stored in the storage 234 and loaded into the memory 233 upon execution of the firmware. Processor 232 may execute firmware loaded into memory 233. The PHR generator 240 may operate to embed the learning EMR EMRa and the learning PHR PHRa under the control of the processor 232, learn the generation model based on this, and generate the virtual PHR. The health predictor 250 may operate to construct a prediction model based on a multi-modality under the control of the processor 232 and analyze the EMR and virtual PHR to generate prediction data. The PHR generator 240 and the health predictor 250 correspond to the PHR generator 240 and the health predictor 250 of FIG. 2, respectively.

Unlike FIG. 6, the PHR generator 240 and the health predictor 250 may be implemented in separate hardware. For example, the PHR generator 240 and the health predictor 250 may be implemented in a neuromorphic chip or the like for constructing a generation model or a prediction model by performing learning through an artificial neural network, or may be implemented in a dedicated logic circuit such as a Field Programmable Gate Array (FPGA) or an Application Specific Integrated Circuit (ASIC).

The storage 234 may store data generated by the operating system or applications for the purpose of long-term storage, a file for running the operating system, or executable files of applications. For example, the storage 234 may store files for execution of the PHR generator 240 and the health predictor 250. The storage 234 may be used as an auxiliary storage device of the medical data processing device 230. The storage 234 may include a flash memory, a phase-change RAM (PRAM), a magnetic RAM (MRAM), a ferroelectric RAM (FeRAM), a resistive RAM (RRAM), and so on.

The bus 235 may provide a communication path between the components of the medical data processing device 130. The network interface 231, the processor 232, the memory 233, and the storage 234 may exchange data with one another through the bus 235. The bus 235 may be configured to support various types of communication formats used in the medical data processing device 230.

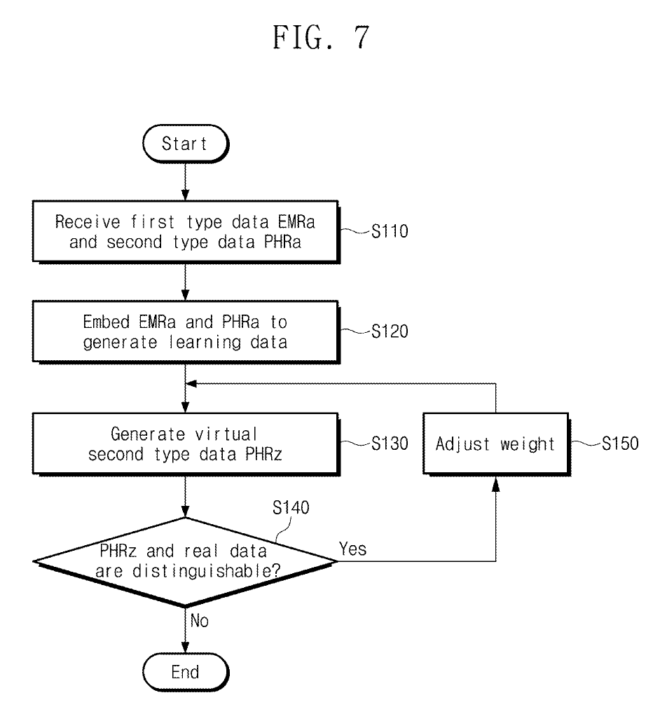

FIG. 7 is a view for explaining a process of learning a generation model by the medical data processing device of FIGS. 2 and 6. Each of the operations of FIG. 7 is performed in the medical data processing device 230 of FIGS. 2 and 6 and may be executed by the processor 232 of FIG. 6. Each of the operations of FIG. 7 may be processed in the PHR generator 240 under the control of the processor 232. For convenience of description, FIG. 7 will be described with reference to the reference numerals of the PHR generator 240a in FIG. 3.

In operation S110, the PHR generator 240a receives the first type data and the second type data through the network interface. The first type data is time series data having a first type, and may be, for example, a learning EMR EMRa. The second type data is time series data having a second type different from the first type, and may be, for example, a learning PHR PHRa. The first and second type data may be provided from a device such as the second collection device 220 of FIG. 2. The first type data and the second type data may be time series data corresponding to past time points, that is, the previous time of the target time point.

In operation S120, the PHR generator 240a may generate the learning data TDa by embedding the first and second type data (i.e., the learning EMR EMRa and the learning PHR PHRa). Operation S120 may be performed in the embedder 241a of the PHR generator 240a. The embedder 241a may embed the first and second type data to have the same type. As a result, the first type data and the second type data may be converted to have the same vector type.

In operation S130, the PHR generator 240a may generate virtual second type data based on the learning data TDa. Operation S130 may be performed in the generator 242a of the PHR generator 240a. The virtual second type data is time series data made to have a second type, and may be, for example, the virtual time series data PHRz in FIG. 3. The generator 242a is implemented with a learnable generation model, and the generation model may generate virtual second type data in response to the input learning data TDa. The virtual second type data may be time series data like the one generated at the previous time of past time points, that is, the target time point.

In operation S140, the PHR generator 240a determines that virtual second type data (i.e., virtual time series data PHRz) is real data RDa. Operation S140 may be performed in the discriminator 243a of the PHR generator 240a. The real data corresponds to the real data RDa described with reference to FIG. 3. When the discriminator 243a may discriminate the virtual second type data and the real data RDa from each other, since the virtual second type data is hardly seen as an actual PHR, operation S150 proceeds. When the discriminator 243a fails to distinguish virtual second type data and real data RDa from each other, the virtual second type data may be regarded as having reliability enough to be seen as an actual PHR. Thus, the operation of learning the generation model is terminated. Then, the virtual PHR generated through the learned generation model may be used for future health prediction.

In operation S150, the weight of the PHR generator 240a is adjusted. It is difficult to see that the current generation model is learned enough to generate time series data with the same reliability as the actually collected PHR. Accordingly, the weight for generating the virtual second type data of the generator 242a is adjusted. Thereafter, operations S130 and S140 are repeated. That is, operations S130 to S150 may be repeated until the PHR generator 240a generates virtual time series data that is difficult to distinguish from the real data RDa.

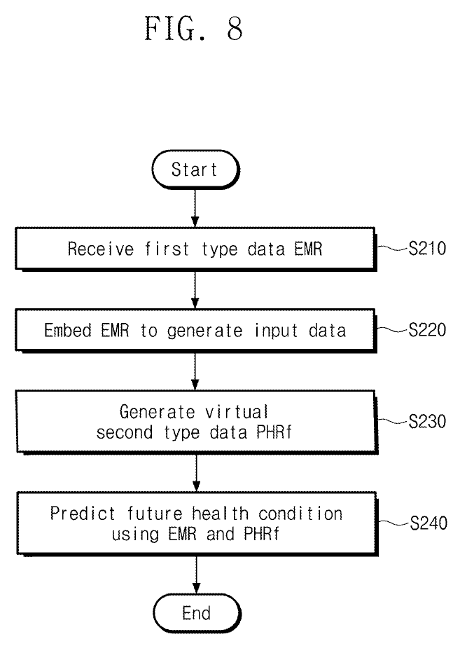

FIG. 8 is a view for explaining a process in which the medical data processing device of FIGS. 2 and 6 operates based on a learned generation model. Each of the operations of FIG. 8 is performed in the medical data processing device 230 of FIGS. 2 and 6 and may be executed by the processor 232 of FIG. 6. Each of the operations of FIG. 8 may be processed in the PHR generator 240 or the health predictor 250 under the control of the processor 232. For convenience of description, FIG. 8 will be described with reference to the reference numerals of the PHR generator 240b in FIG. 4.

In operation S210, the PHR generator 240b receives the first type data through the network interface. The first type data may be time series data having a first type, for example, an EMR provided from the first collection device 210 of FIG. 2. The first type data may be time series data corresponding to the previous time of past time points, that is, the target time point.

In operation S220, the PHR generator 240b may generate input data ID by embedding the first type data (i.e., EMR). Operation S220 may be performed in the embedder 241b of the PHR generator 240b. In operation S120 of FIG. 7, the embedder 241b may convert the EMR so that the first and second type data have the same vector type as the converted vector type.

In operation S230, the PHR generator 240b may generate virtual second type data based on the input data ID. Operation S230 may be performed in the generator 242b of the PHR generator 240b. The virtual second type data is time series data made to have a second type, and may be, for example, the virtual time series data PHRz in FIG. 4. Through the learning operations of FIG. 7, in response to the input data ID, the generated generation model may generate virtual second type data that is the same as that generated at the previous time of past time points, that is, the target time point.

In operation S240, the health predictor 250 included in the medical data processing device 230 may predict a future health condition based on first type data (i.e., EMR) and virtual second type data (i.e., virtual PHR PHRf). The health predictor 250 may generate prediction data corresponding to a time after a future time point, i.e., a target time point, based on the first type data and the virtual second type data. The prediction data is not limited, but may be the predicted EMR of the future time point. The health predictor 250 may be implemented with a multi-modality based prediction model. Illustratively, in operation S240, a first intermediate data may be generated based on a time series transition of the first type data, and second intermediate data may be generated based on time series transition of the virtual second type data. The health predictor 250 may calculate the prediction data based on the first and second intermediate data.

The time series data processing device, the health prediction system including the same, and the method for operating the time series data processing device according to an embodiment of the inventive concept may use a prediction model for analyzing time series data having different types or modalities, so that the prediction accuracy for the time point may be improved.

In addition, the time series data processing device, the health prediction system including the same, and the method for operating the time series data processing device according to an embodiment of the inventive concept may generates virtual time series data having a specified type, so that it may utilize the prediction model that is already constructed even in the absence or lack of time series data, and may reduce the collection burden of time series data.

Although the exemplary embodiments of the inventive concept have been described, it is understood that the inventive concept should not be limited to these exemplary embodiments but various changes and modifications may be made by one ordinary skilled in the art within the spirit and scope of the inventive concept as hereinafter claimed.

What is claimed is:

1. A time series data processing device comprising:
a network interface configured to receive first time series data corresponding to a previous time of a target time point, the first time series data having a first type;
a data generator configured to generate a second time series data corresponding to a previous time of the target time point based on the first time series data, the second time series data having a second type;
a predictor configured to generate prediction data corresponding to a later time of the target time point based on the first time series data and the second time series data; and
a processor configured to control the data generator and the predictor.

2. The device of claim 1, wherein the first time series data is a grouped electronic medical record generated at a plurality of time points preceding the target time point,
wherein the data generator generates the second time series data corresponding to a virtual personal health record based on the electronic medical record.

3. The device of claim 1, wherein the data generator generates the second time series data based on a generation model learned by third time series data having the first type and fourth time series data having the second type,
wherein the network interface receives the third and fourth time series data before receiving the first time series data.

4. The device of claim 3, wherein the data generator comprises:
a generator configured to generate fifth time series data having the second type based on the third and fourth time series data; and
a discriminator configured to determine whether the fifth time series data is data generated from the generator.

5. The device of claim 4, wherein until the discriminator does not determine the fifth time series data as data generated from the generator, a weight of the generation model is adjusted.

6. The device of claim 3, wherein the data generator comprises:
an embedder configured to convert each of the third time series data and the fourth time series data to have the same type,
wherein the generation model is learned based on the converted third and fourth time series data.

7. The device of claim 6, wherein the embedder converts the first time series data to have the same type as the converted third and fourth time series data,
wherein the generation model generates the second time series data based on the converted first time series data.

8. The device of claim 1, wherein the first time series data comprises first feature data that is numerical data and second feature data that is non-numerical data,
wherein the data generator converts the second feature data into numerical data and generates the second time series data based on the first feature data and the second feature data converted into the numerical data.

9. The device of claim 1, wherein the second time series data is time series data having a predetermined reference time interval.

10. A health prediction system comprising:
a collection device configured to collect first time series data corresponding to an electronic medical record; and
a medical data processing device configured to generate second time series data corresponding to a virtual personal health record and having a reference time interval based on the first time series data, and generate prediction data of a future time point based on the first time series data and the second time series data.

11. The system of claim 10, wherein the medical data processing device comprises:
a personal health record generator configured to generate the second time series data based on the first time series data; and
a health predictor configured to generate the electronic medical record of the future time point based on the first and second time series data.

12. The system of claim 11, wherein the health predictor generates the prediction data corresponding to the electronic medical record of the future time point, based on a prediction model for analyzing a change trend of the first time series data with respect to time and a change trend of the second time series data with respect to time in parallel.

13. The system of claim 10, further comprising a second collection device configured to collect third time series data corresponding to the second electronic medical record and a fourth time series data corresponding to a personal health record measured from a personal health sensor,
wherein the medical data processing device learns a generation model based on the third and fourth time series data and inputs the first time series data to the generation model to generate the second time series data.

14. The system of claim 13, wherein the medical data processing device inputs the third and fourth time series data to the generation model to generate fifth time series data corresponding to a virtual personal health record, and learns the generation model until it is not determined whether the fifth time series data is the virtual personal health record or the measured personal health record.

15. The system of claim 13, wherein the medical data processing device converts each of the third time series data and the fourth time series data to have the same type and inputs the converted third and fourth time series data to the generation model.

16. A method of operating a time series data processing device performed by a processor, the method comprising:
receiving first time series data generated to have a first type at past time points, through a network interface;
embedding the first time series data to generate input data;
inputting the input data to a generation model to generate second time series data corresponding to past time points having a reference time interval and having a second type; and
generating prediction data of a future time point based on the first time series data and the second time series data.

17. The method of claim 16, further comprising, before receiving the first time series data, learning the generation model, based on third time series data collected to have the first type and fourth time series data collected to have the second type.

18. The method of claim 17, wherein the learning of the generation model comprises:
receiving the third and fourth time series data through the network interface;
generating learning data by embedding the third and fourth time series data to have the same type;
inputting the learning data to the generation model to generate fifth time series data corresponding to past time points having the reference time interval and having the second type; and
determining whether the fifth time series data is time series data received through the network interface or time series data generated from the generation model.

19. The method of claim 18, wherein the learning of the generation model further comprises, when the fifth time series data is determined as time series data generated from the generation model, adjusting a weight of the generation model.

20. The method of claim 16, wherein the generating of the prediction data comprises:
generating first intermediate data based on a change trend of the first time series data with respect to time;
generating second intermediate data based on a change trend of the second time series data with respect to time; and
calculating the prediction data based on the first intermediate data and the second intermediate data.
